(19) United States
(12) Patent Application Publication
(10) Pub. No.: US 2010/0275931 A1
(43) Pub. Date: Nov. 4, 2010

(54) Earplug For An Ear Canal

(76) Inventor: Saeed Seyed, Los Altos, CA (US)

Correspondence Address: Saeed Seyed, 25525 Adobe Lane, Los Altos, CA, 94022, US

(21) Appl. No.: 12/434,611

(22) Filed: May 1, 2009

(51) Int. Cl. A61F 11/08

(52) U.S. Cl. 128/864

(57) ABSTRACT

An earplug for an ear canal includes a cap for plugging the ear canal. The cap has a cover tapered at one end and a hollow post extending centrally from the end. An air gap is formed between the cover and the post. The cover further includes a flexible rubbery material that adjusts shape to match a shape of the ear canal and seal the ear canal. An inner spring holds the cap in the ear canal. The inner spring has a plurality of spring leafs about a central axis to fit within the hollow post where the spring leafs are compressed about the central axis and a force of the spring leafs flexes the cap to hold the cap in the ear canal. A base is joined to an end of the inner spring for gripping the earplug for insertion and removal of the earplug from the ear canal.

### FEDERALLY SPONSORED RESEARCH OR DEVELOPMENT

Not applicable.

### REFERENCE TO SEQUENCE LISTING, A TABLE, OR A COMPUTER LISTING APPENDIX

Not applicable.

### COPYRIGHT NOTICE

A portion of the disclosure of this patent document contains material that is subject to copyright protection. The copyright owner has no objection to the facsimile reproduction by anyone of the patent document or patent disclosure as it appears in the Patent and Trademark Office, patent file or records, but otherwise reserves all copyright rights whatsoever.

### FIELD OF THE INVENTION

The present invention relates generally to earplugs. More particularly, the invention relates to an earplug for swimming with a mechanical spring that provides a solid grip by compression force and generally prevents water from getting into the ear.

### BACKGROUND OF THE INVENTION

Earplugs are often worn by swimmers to prevent water from getting in their ears. The earplugs in market today have two major problems. Wax type earplugs are easy to use and are effective at preventing water from entering the ear. However, wax type earplugs may crack or break into pieces after being used a few times. This may cause pieces of the earplug to get inside the ear, which is hard to get out. Other types of earplugs do not have a good grip inside the ear and can be potentially lost while swimming. Other problems that arise from currently known earplugs are that some may be uncomfortable in the ear and some are difficult to use.

In view of the foregoing, there is a need for improved techniques for providing earplugs for swimming that effectively remain in the ears and prevent water from entering the ears, do not crack, are comfortable, and are easy to use.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1 is an exploded view of an exemplary earplug comprising a mechanical spring, in accordance with an embodiment of the present invention;

FIGS. 2A and 2B illustrate an exemplary assembled earplug comprising a mechanical inner spring, in accordance with an embodiment of the present invention. FIG. 2A is a front perspective view, and FIG. 2B is a rear perspective view;

FIGS. 3A and 3B illustrate an exemplary outer cap of an earplug comprising a mechanical inner spring, in accordance with an embodiment of the present invention. FIG. 3A is a front perspective view, and FIG. 3B is a rear perspective view; and

FIGS. 4A and 4B illustrate an exemplary inner spring of an earplug comprising a mechanical inner spring, in accordance with an embodiment of the present invention. FIG. 4A is a front perspective view, and FIG. 4B is a rear perspective view.

Unless otherwise indicated illustrations in the figures are not necessarily drawn to scale.

### SUMMARY OF THE INVENTION

To achieve the forgoing and other objects and in accordance with the purpose of the invention, an earplug for an ear canal is presented.

In one embodiment, an earplug for an ear canal is presented. The earplug includes means for plugging the ear canal and means for holding the plugging means in the ear canal.

In another embodiment an earplug for an ear canal is presented. The earplug includes a cap for plugging the ear canal. The cap has a tapered cover and a hollow post extending centrally from the cover where an air gap is formed between the cover and the post. An inner spring holds the cap in the ear canal. The inner spring has a plurality of spring leafs dimensioned to fit in the hollow post where the spring leafs are compressed and a force of the spring leafs holds the cap in the ear canal.

In another embodiment an earplug for an ear canal is presented. The earplug includes a cap for plugging the ear canal. The cap has a cover tapered at one end and a hollow post extending centrally from the one end. An air gap is formed between the cover and the post. The cover further includes a flexible rubbery material that adjusts shape to substantially match a shape of the ear canal and substantially seal the ear canal. An inner spring holds the cap in the ear canal. The inner spring has a plurality of spring leafs about a central axis and dimensioned to fit within the hollow post where the spring leafs are compressed about the central axis and a force of the spring leafs flexes the cap to hold the cap in the ear canal. A base is joined to an end of the inner spring for gripping the earplug for insertion and removal of the earplug from the ear canal.

Other features, advantages, and object of the present invention will become more apparent and be more readily understood from the following detailed description, which should be read in conjunction with the accompanying drawings.

### DETAILED DESCRIPTION OF THE PREFERRED EMBODIMENTS

The present invention is best understood by reference to the detailed figures and description set forth herein.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are numerous modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

The present invention will now be described in detail with reference to embodiments thereof as illustrated in the accompanying drawings.

Preferred embodiments of the present invention provide an earplug for swimming with a mechanical spring that allows for a solid grip by compression force and generally prevents water from getting into the ear. Preferred embodiments comprise two parts. An inner part is a mechanical spring that is adjustable in diameter. This makes the earplug easy to fit and mechanically holds the earplug in place. The second part is a flexible and comfortable rubber piece that plugs into the ear to generally prevent water from getting into ear. In preferred embodiments these two parts are attached together to form the earplug.

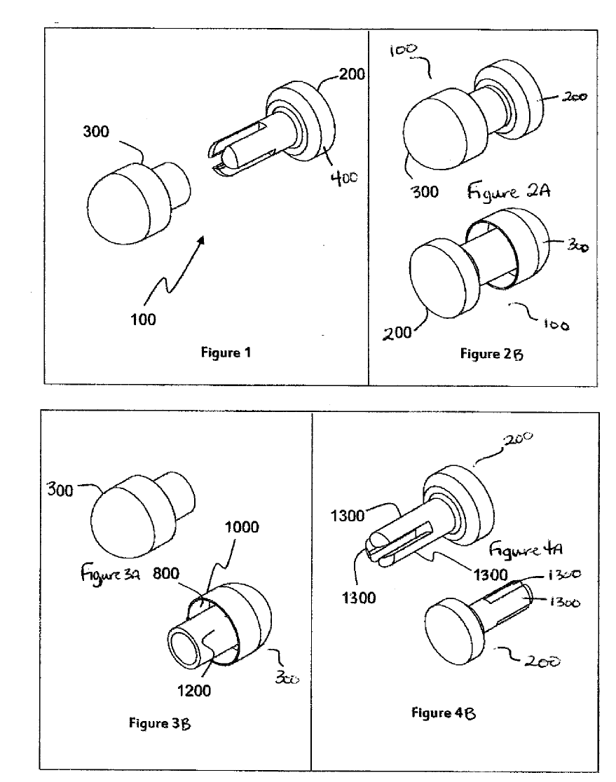

FIG. 1 is an exploded view of an exemplary earplug 100 comprising a mechanical inner spring 200, in accordance with an embodiment of the present invention. In the present embodiment, earplug 100 is configured with inner spring 200 and a cap 300. In the present embodiment, inner spring 200 adjusts in size so that one size fits all adults. However, earplugs according to some embodiments of the present invention may be available in various sizes to accommodate different ear sizes. At the time of inserting earplug 100 into an ear, inner spring 200 compresses to a smaller diameter, and once earplug 100 is fitted inside the ear, inner spring 200 expands back to its original diameter to fit tightly inside the ear. Cap 300 is smooth on the surface and flexible enough to seal the ear canal. Cap 300 is preferably covered in a comfortable, flexible material such as, but not limited to, rubber, silicone, various plastics, etc. What distinguishes the present embodiment from other designs is its combination of mechanical inner spring 200 and comfortable, rubbery material on cap 300.

In typical use of the present embodiment, earplug 100 is adjustable in diameter as it is being plugged into an ear, similar to a soft spring. Earplug 100 adjusts its shape to the shape of the ear with flexible rubber cap 300 and the geometry and spring design of inner spring 200 to prevent water from getting into the ear. Earplug 100 plugs into the ear easily and comfortably and is retained comfortably inside the ear and will not become loose or lost during swimming because of the spring factor of the present embodiment. Furthermore, the spring design and an easily gripped base 400 of inner spring 200 enable earplug 100 to be removed easily from the ear. The construction of the present embodiment enables earplug 100 to be very durable so that it may be used and reused numerous times.

FIGS. 2A and 2B illustrate an exemplary assembled earplug 100 comprising a mechanical inner spring 200, in accordance with an embodiment of the present invention. FIG. 2A is a front perspective view, and FIG. 2B is a rear perspective view. In the present embodiment, inner spring 200 is inserted into an outer cap 300. Earplug 100 is assembled in this manner before being inserted into an ear. Alternate embodiments may comprise inner springs and outer caps in various shapes and sizes that may be mixed and matched to create earplugs to fit ears of many different sizes and shapes. In some embodiments, the earplugs may be made in many colors to appeal to a wide rage of users, and other embodiments may be transparent or flesh colored to blend in with the ear.

FIGS. 3A and 3B illustrate an exemplary cap 300 of an earplug comprising a mechanical inner spring, in accordance with an embodiment of the present invention. FIG. 3A is a front perspective view, and FIG. 3B is a rear perspective view. In the present embodiment, cap 300 comprises a cover 1000 that is tapered and a post 1200. An air gap 800 between cover 1000 and post 1200 enables cover 1000 to flex to easily be slid into the ear. As cap 300 is guided into the ear, cover 1000 moves toward post 1200 and plugs the ear.

FIGS. 4A and 4B illustrate an exemplary inner spring 200 of an earplug comprising mechanical inner spring 200, in accordance with an embodiment of the present invention. FIG. 4A is a front perspective view, and FIG. 4B is a rear perspective view. In the present embodiment, inner spring 200 comprises leafs 1300. As an earplug comprising inner spring 200 slides into an ear, spring leafs 1300 compress to the ear size, and once the earplug is in place, spring leafs 1300 expand and hold the earplug in place by compression force.

Preferred embodiments of the present invention are made for use during swimming. However, alternate embodiments may be implemented for use in normal headphones. Current earbud headphones do not have a spring factor and can drop from a user's ears very easily. Incorporating an inner spring according to an embodiment of the present invention will more securely hold the headphones in the user's ears

Having fully described at least one embodiment of the present invention, other equivalent or alternative methods of providing an earplug comprising a mechanical spring according to the present invention will be apparent to those skilled in the art. The invention has been described above by way of illustration, and the specific embodiments disclosed are not intended to limit the invention to the particular forms disclosed. For example, the particular implementation of the cap may vary depending upon the particular preference of the user. The caps described in the foregoing were directed to rounded, tapered cylindrical implementations; however, similar techniques are to provide caps in various different shapes such as, but not limited to, cones, non-tapered cylinders, spheres, irregular shapes to match an ear canal, etc. Implementations of the present invention that comprise caps of different shapes are contemplated as within the scope of the present invention. The invention is thus to cover all modifications, equivalents, and alternatives falling within the spirit and scope of the following claims.

What is claimed is:

1. An earplug for an ear canal, the earplug comprising:
means for plugging the ear canal; and
means for holding said plugging means in the ear canal.

2. The earplug as recited in claim 1, further comprising means for gripping the earplug.

3. An earplug for an ear canal, the earplug comprising:
a cap for plugging the ear canal, said cap having a tapered cover and a hollow post extending centrally from said cover where an air gap is formed between said cover and said post; and
an inner spring for holding said cap in the ear canal, said inner spring having a plurality of spring leafs dimensioned to fit in said hollow post where said spring leafs are compressed and a force of said spring leafs holds said cap in the ear canal.

4. The earplug as recited in claim 3, further comprising a base joined to an end of said inner spring for gripping the earplug.

5. The earplug as recited in claim 3, wherein said tapered cover comprises a flexible material that substantially seals the ear canal.

6. The earplug as recited in claim 5, wherein said flexible material further comprises rubber or silicon.

7. The earplug as recited in claim 3, wherein the earplug is transparent or flesh colored to blend in with an ear.

8. The earplug as recited in claim 3, wherein the earplug is adapted for use as a headphone.

9. An earplug for an ear canal, the earplug comprising:
a cap for plugging the ear canal, said cap having a cover tapered at one end and a hollow post extending centrally from said one end where an air gap is formed between said cover and said post, said cover further comprising a flexible rubbery material that adjusts shape to substantially match a shape of the ear canal and substantially seal the ear canal;
an inner spring for holding said cap in the ear canal, said inner spring having a plurality of spring leafs about a central axis and dimensioned to fit within said hollow post where said spring leafs are compressed about said central axis and a force of said spring leafs flexes said cap to hold said cap in the ear canal; and
a base joined to an end of said inner spring for gripping the earplug for insertion and removal of the earplug from the ear canal.

10. The earplug as recited in claim 9, wherein the earplug is adapted for use as an earbud headphone.
